(19) United States
(12) Patent Application Publication
(10) Pub. No.: US 2012/0238849 A1
(43) Pub. Date: Sep. 20, 2012

(54) INFUSION SET COMPONENT WITH INTEGRATED ANALYTE SENSOR CONDUCTORS

(75) Inventors: Kris R. HOLTZCLAW, Santa Clarita, CA (US); Fan MENG, San Marino, CA (US)

(73) Assignee: MEDTRONIC MINIMED, INC., Northridge, CA (US)

(21) Appl. No.: 13/050,814

(22) Filed: Mar. 17, 2011

(51) Int. Cl. A61B 5/145; A61M 5/168

(52) U.S. Cl. 600/345; 604/66

(57) ABSTRACT

An infusion set component for a fluid infusion device that delivers fluid to a patient is presented here. The component includes a tube formed from tubing material having an interior fluid canal defined therein to provide a fluid pathway from the fluid infusion device to the patient, a plurality of sensor conductors incorporated with the tubing material to facilitate sensing of an analyte of the patient by the fluid infusion device, and a combined infusion-sensor unit coupled to the tube and to the plurality of sensor conductors. The infusion-sensor unit accommodates delivery of fluid from the tube, and it also accommodates sensing of the analyte. The component may also include a connector assembly coupled to the tube and to the plurality of sensor conductors, to fluidly couple the fluid canal to a fluid reservoir of the fluid infusion device and to electrically couple the sensor conductors to an electronics module of the fluid infusion device.

### TECHNICAL FIELD

Embodiments of the subject matter described herein relate generally to fluid infusion devices, such as insulin pumps. More particularly, embodiments of the subject matter relate to an infusion set component having analyte sensor conductors integrated into the infusion tubing material.

### BACKGROUND

Portable medical devices are useful for patients that have conditions that must be monitored on a continuous or frequent basis. For example, diabetics are usually required to modify and monitor their daily lifestyle to keep their blood glucose (BG) in balance. Individuals with Type 1 diabetes and some individuals with Type 2 diabetes use insulin to control their BG levels. To do so, diabetics routinely keep strict schedules, including ingesting timely nutritious meals, partaking in exercise, monitoring BG levels daily, and adjusting and administering insulin dosages accordingly.

The prior art includes a number of fluid infusion devices and insulin pump systems that are designed to deliver accurate and measured doses of insulin via infusion sets (an infusion set delivers the insulin through a small diameter tube that terminates at, e.g., a cannula inserted under the patient's skin). In lieu of a syringe, the patient can simply activate the insulin pump to administer an insulin bolus as needed, for example, in response to the patient's high BG level.

A typical infusion pump includes a housing, which encloses a pump drive system, a fluid containment assembly, an electronics system, and a power supply. The pump drive system typically includes a small motor (DC, stepper, solenoid, or other varieties) and drive train components such as gears, screws, and levers that convert rotational motor motion to a translational displacement of a stopper in a reservoir. The fluid containment assembly typically includes the reservoir with the stopper, tubing, and a catheter or infusion set to create a fluid path for carrying medication from the reservoir to the body of a user. The electronics system regulates power from the power supply to the motor. The electronics system may include programmable controls to operate the motor continuously or at periodic intervals to obtain a closely controlled and accurate delivery of the medication over an extended period.

The prior art also includes a variety of physiological characteristic (or analyte) sensors that are designed to measure an analyte of a patient. For example, continuous glucose sensors employ a subcutaneous glucose sensor technology that facilitates ongoing monitoring of blood glucose levels. Continuous glucose sensors may utilize wireless data communication techniques to transmit data indicative of the blood glucose levels to a portable infusion pump, a glucose monitor device, and/or other receiving devices. Thus, in a typical insulin pump system, the patient might wear both an infusion set (for the delivery of insulin) and a glucose sensor-transmitter.

### BRIEF SUMMARY

An exemplary embodiment of an infusion set component for a fluid infusion device that delivers fluid to a patient is provided. The infusion set component includes tubing material having an interior fluid canal defined therein to provide a fluid pathway from the fluid infusion device to the patient, and a plurality of sensor conductors integrated with the tubing material to facilitate sensing of an analyte of the patient by the fluid infusion device.

Also provided is another exemplary embodiment of an infusion set component. The infusion set component includes: a tube formed from tubing material having an interior fluid canal defined therein to provide a fluid pathway from the fluid infusion device to the patient; a plurality of sensor conductors incorporated with the tubing material to facilitate sensing of an analyte of the patient by the fluid infusion device; and a combined infusion-sensor unit coupled to the tube and to the plurality of sensor conductors. The combined infusion-sensor unit accommodates delivery of fluid from the interior fluid canal of the tube and accommodating sensing of the analyte.

Yet another exemplary embodiment of an infusion set component is presented here. The infusion set component includes: a tube formed from tubing material having an interior fluid canal defined therein to provide a fluid pathway from the fluid infusion device to the patient; a plurality of sensor conductors molded within in the tubing material to facilitate sensing of an analyte of the patient by the fluid infusion device; and a connector assembly coupled to the tube and to the plurality of sensor conductors. The connector assembly fluidly couples the interior fluid canal to a fluid reservoir of the fluid infusion device and electrically couples the plurality of sensor conductors to an electronics module of the fluid infusion device.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### BRIEF DESCRIPTION OF THE DRAWINGS

A more complete understanding of the subject matter may be derived by referring to the detailed description and claims when considered in conjunction with the following figures, wherein like reference numbers refer to similar elements throughout the figures.

FIG. 1 is a perspective view of an exemplary embodiment of a fluid infusion system;

FIG. 2 is a cross-sectional view of an exemplary embodiment of an infusion set component suitable for use with the fluid infusion system shown in FIG. 1;

FIG. 3 is a cross-sectional view of an exemplary concentric tube embodiment of an infusion set component suitable for use with the fluid infusion system shown in FIG. 1;

FIG. 4 is a cross-sectional view of another exemplary embodiment of an infusion set component suitable for use with the fluid infusion system shown in FIG. 1;

FIG. 5 is a cross-sectional view of yet another exemplary embodiment of an infusion set component suitable for use with the fluid infusion system shown in FIG. 1;

FIG. 6 is a diagram that illustrates a twisted arrangement of sensor conductors along the length of an infusion set component;

FIG. 7 is a side view of an exemplary embodiment of a combined infusion-sensor unit suitable for use with the fluid infusion system shown in FIG. 1;

FIG. 8 is a schematic representation of electrodes of an analyte sensor suitable for use with the fluid infusion system shown in FIG. 1;

FIG. 9 is a schematic representation of electrical contacts of a reservoir cap suitable for use with the fluid infusion system shown in FIG. 1;

FIG. 10 is a schematic representation of a fluid infusion device configured in accordance with one exemplary embodiment; and

FIG. 11 is a schematic representation of a fluid infusion device configured in accordance with another exemplary embodiment.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Any implementation described herein as exemplary is not necessarily to be construed as preferred or advantageous over other implementations. Furthermore, there is no intention to be bound by any expressed or implied theory presented in the preceding technical field, background, brief summary or the following detailed description.

Techniques and technologies may be described herein in terms of functional and/or logical block components, and with reference to symbolic representations of operations, processing tasks, and functions that may be performed by various computing components or devices. It should be appreciated that the various block components shown in the figures may be realized by any number of hardware, software, and/or firmware components configured to perform the specified functions. For example, an embodiment of a system or a component may employ various integrated circuit components, e.g., memory elements, digital signal processing elements, logic elements, look-up tables, or the like, which may carry out a variety of functions under the control of one or more microprocessors or other control devices.

The subject matter described here relates to a fluid infusion device of the type used to treat a medical condition of a patient. The infusion device is used for infusing fluid into the body of a user. The non-limiting examples described below relate to a medical device used to treat diabetes (more specifically, an insulin pump), although embodiments of the disclosed subject matter are not so limited. Accordingly, the infused fluid is insulin in certain embodiments. In alternative embodiments, however, many other fluids may be administered through infusion such as, but not limited to, disease treatments, drugs to treat pulmonary hypertension, iron chelation drugs, pain medications, anti-cancer treatments, medications, vitamins, hormones, or the like.

For the sake of brevity, conventional features and technologies related to infusion system operation, insulin pump and/or infusion set operation, blood glucose sensing and monitoring, sensor signal processing, and other functional aspects of the fluid infusion system (and the individual operating components of the system) may not be described in detail here. Examples of infusion pumps and/or related pump drive systems used to administer insulin and other medications may be of the type described in, but not limited to, U.S. Pat. Nos. 4,562,751; 4,678,408; 4,685,903; 5,080,653; 5,505,709; 5,097,122; 6,485,465; 6,554,798; 6,558,351; 6,659,980; 6,752,787; 6,817,990; 6,932,584; and 7,621,893; which are herein incorporated by reference.

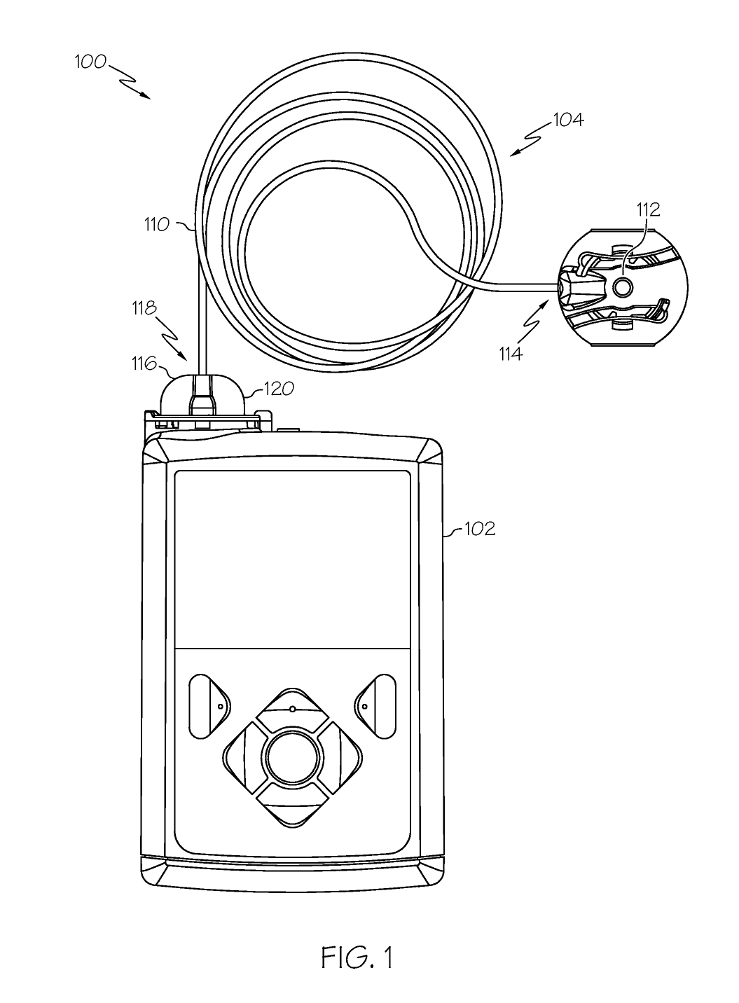

FIG. 1 is a perspective view of an exemplary embodiment of a fluid infusion system 100. The system 100 includes two main components: a fluid infusion device 102 (e.g., an insulin pump) and an infusion set component 104, which can be coupled to the fluid infusion device 102 as depicted in FIG. 1. This particular embodiment of the infusion set component 104 includes, without limitation: a tube 110; a combined infusion-sensor unit 112 coupled to one end 114 of the tube 110; and a connector assembly 116 coupled to the other end 118 of the tube 110. The fluid infusion device 102 is designed to be carried or worn by the patient, and the infusion set component 104 terminates at the combined infusion-sensor unit 112 such that the fluid infusion device 102 can deliver fluid to the body of the patient via the tube 110. Moreover, the combined infusion-sensor unit 112 cooperates with the fluid infusion device 102 to sense, measure, or detect an analyte of the patient (such as blood glucose), as described in more detail below. The fluid infusion device 102 may leverage a number of conventional features, components, elements, and characteristics of existing fluid infusion devices. For example, the fluid infusion device 102 may incorporate some of the features, components, elements, and/or characteristics described in U.S. Pat. Nos. 6,485,465 and 7,621,893, the relevant content of which is incorporated by reference herein.

The fluid infusion device 102 accommodates a fluid reservoir (hidden from view in FIG. 1) for the fluid to be delivered to the user. The tube 110 represents the fluid flow path that couples the fluid reservoir to the combined infusion-sensor unit 112. When installed as depicted in FIG. 1, the tube 110 extends from the fluid infusion device 102 to the combined infusion-sensor unit 112, which in turn provides a fluid pathway to the body of the patient. For the illustrated embodiment, the connector assembly 116 is realized as a removable reservoir cap 120 (or fitting) that is suitably sized and configured to accommodate replacement of fluid reservoirs (which are typically disposable) as needed. In this regard, the reservoir cap 120 is designed to accommodate the fluid path from the fluid reservoir to the tube 110.

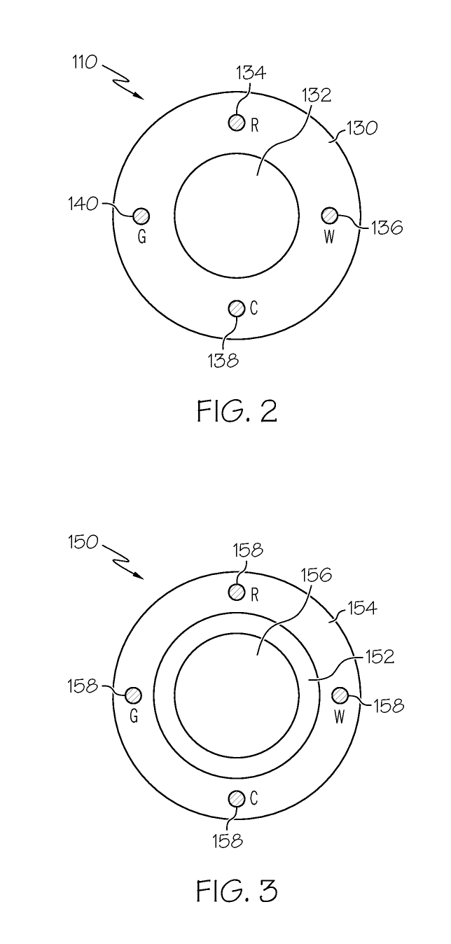

The tube 110 is fabricated with electrical sensor conductors integrated therewith to support the operation of an analyte sensor located at the combined infusion-sensor unit 112. The sensor conductors facilitate sensing of an analyte of the patient (e.g., blood glucose) by the fluid infusion device 102, which may apply or detect sensor voltages and/or currents using the sensor conductors. In this regard, FIG. 2 is a cross-sectional view of an exemplary embodiment of the tube 110. The tube 110 is formed from an appropriate type and composition of tubing material 130, which is fabricated with an interior fluid canal 132 defined therein. The interior fluid canal 132 provides a fluid pathway from the fluid infusion device 102 to the patient. In other words, the interior fluid canal 132 is present throughout the length of the tube 110 (i.e., the major dimension of the tube 110). In a typical implementation, the tube 110 has an outer diameter within the range of about 0.060±0.001 inches, and the interior fluid canal 132 has a diameter within the range of about 0.016±0.001 inches. The tubing material 130 may be any flexible, tough, and lightweight material such as, without limitation: a polyethylene polymer; a polyurethane polymer; or the like. For the exemplary embodiment described here, the tubing material 130 is a molded or extruded concentric construction, where an inner tube is formed from a polyethylene polymer and an outer tube is formed from a polyurethane polymer. Alternatively, the tube 110 could be fabricated as a single tube construction.

The embodiment depicted in FIG. 2 includes four sensor conductors embedded in the tubing material 130, although alternate embodiments may include more or less than four sensor conductors. The sensor conductors may be realized as thin cooper wires, metal traces, or conductive filaments. The sensor conductors are embedded such that the tubing material 130 surrounds, encases, and insulates each of the individual sensor conductors. In this regard, the tubing material 130 may be composed of an electrically insulating material to electrically insulate each of the sensor conductors. In such an embodiment, the sensor conductors need not be individually surrounded by an insulating sleeve or casing. In practice, the sensor conductors could be molded within the tubing material 130 such that they are spaced apart from one another as shown in the cross-sectional view of FIG. 2.

For consistency with certain legacy sensor technologies, the tube 110 has the following sensor conductors embedded therein: a reference conductor 134; a working conductor 136; and a counter conductor 138. Indeed, certain embodiments of the tube 110 may include only these three conductors. The illustrated embodiment of the tube 110, however, also includes an embedded ground conductor 140. The reference conductor 134 is used for, corresponds to, and is coupled to a reference electrode of the analyte sensor (which forms a part of the combined infusion-sensor unit 112). Similarly: the working conductor 136 is used for, corresponds to, and is coupled to a reference electrode of the analyte sensor; and the counter conductor 138 is used for, corresponds to, and is coupled to a counter electrode of the analyte sensor. The reference electrode, the working electrode, and the counter electrode are utilized to measure the desired analyte level, in accordance with conventional techniques and principles. In certain implementations, the ground conductor 140 could be used to support additional functionality that need not relate to the core function of the analyte sensor. For example, the ground conductor 140 may be used to implement a micro-fuse feature that is “blown” after the combined infusion-sensor unit 112 has been in use longer than its recommended time period. Thus, the ground conductor 140 could be utilized for any desired feature or function that requires or relies on an electrical ground connection.

The embodiment depicted in FIG. 3 employs an exemplary concentric tube construction that is similar to the tube 110. The tube 150 shown in FIG. 3 includes an inner tube 152 and an outer tube 154 that is concentric with the inner tube 152. Thus, the inner tube 152 defines the interior fluid canal 156 of the tube 150, and the outer tube 154 surrounds the inner tube 152. Although not always required, the tubing material of the inner tube 152 is different than the tubing material of the outer tube 154 in this particular embodiment. More specifically, the tubing material of the inner tube 152 may be formed from a polyethylene polymer, while the tubing material of the outer tube 154 may be formed from a polyurethane polymer.

For this exemplary embodiment, the sensor conductors 158 (for consistency with FIG. 2, four conductors are shown in FIG. 3) are molded within, incorporated into, or embedded in the outer tube 154. Alternatively, the sensor conductors may be located in the tubing material of the inner tube 152. In yet other embodiments, the sensor conductors 158 could be positioned in both the inner tube 152 and the outer tube 154. Moreover, it may be desirable or possible to have a single sensor conductor 158 traverse the boundary of the inner tube 152 and the outer tube 154 as it runs along the length of the tube 150. In other words, one or more sections of one sensor conductor 158 might be located in the inner tube 152, while at least one other section of the same sensor conductor 158 might be located in the outer tube 154.

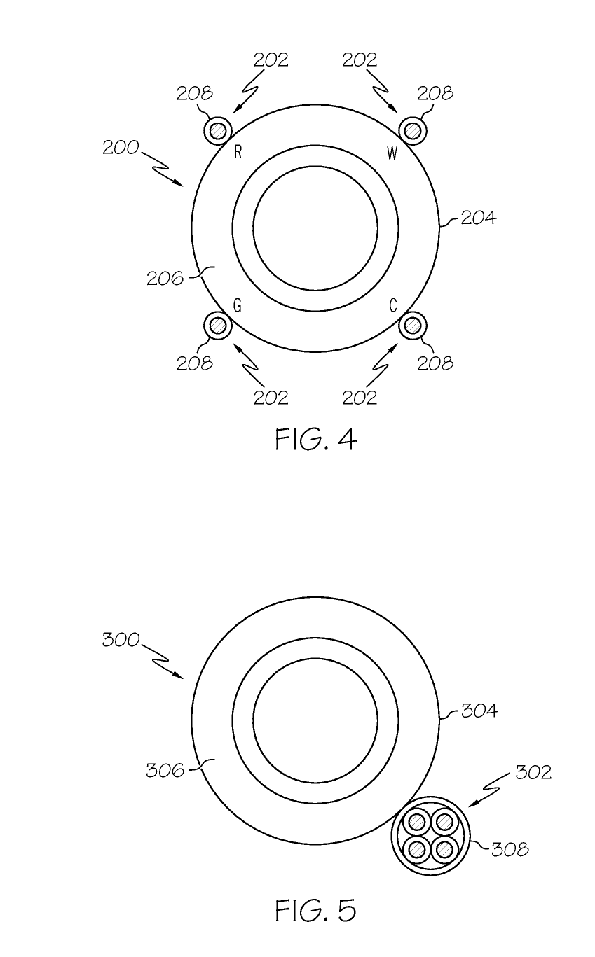

The sensor conductors may be incorporated with the tubing material using other approaches. For example, FIG. 4 is a cross-sectional view of another exemplary embodiment of an infusion set tube 200 having a plurality of sensor conductors 202 incorporated therein. In contrast to the embedded approach shown in FIG. 2, the tube 200 includes the sensor conductors 202 located around the exterior 204 of the tubing material 206 (which may be a single type of material, an inner-outer construction as shown, or any multiple material construction). The sensor conductors 202 may be coupled or attached to the exterior 204 of the tubing material 206, or they may be molded with the tubing material 206 in the form of “appendages” to the tube 200. FIG. 4 depicts an implementation where each sensor conductor 202 includes an outer insulator layer 208 that is distinct and separate from the tubing material 206. Accordingly, the sensor conductors 202 could be fabricated separately as individually insulated wires, and thereafter affixed to the exterior 204 of the tubing material 206 as needed.

FIG. 5 is a cross-sectional view of yet another exemplary embodiment of an infusion set tube 300 having a cluster of sensor conductors 302 integrated therewith. The tube 300 is similar to the tube 200 in that the sensor conductors 302 are located around the exterior 304 of the tubing material 306 (which may be a single type of material, an inner-outer construction as shown, or any multiple material construction). The cluster of sensor conductors 302 may be coupled or attached to the exterior 304 of the tubing material 306, or they may be molded with the tubing material 306 in the form of an “appendage” to the tube 300. FIG. 5 depicts an implementation where each sensor conductor 202 includes a respective outer insulator layer, which insulates each sensor conductor 202 from its neighboring sensor conductors 202. Moreover, FIG. 5 depicts an embodiment where the cluster of sensor conductors 202 are enclosed by an outer layer 308, which is distinct and separate from the tubing material 306. Accordingly, the cluster of sensor conductors 302 could be fabricated separately, with the outer layer 308 holding them as a single cable construction. Thereafter, this combined construction may be affixed to the exterior 304 of the tubing material 306 as needed.

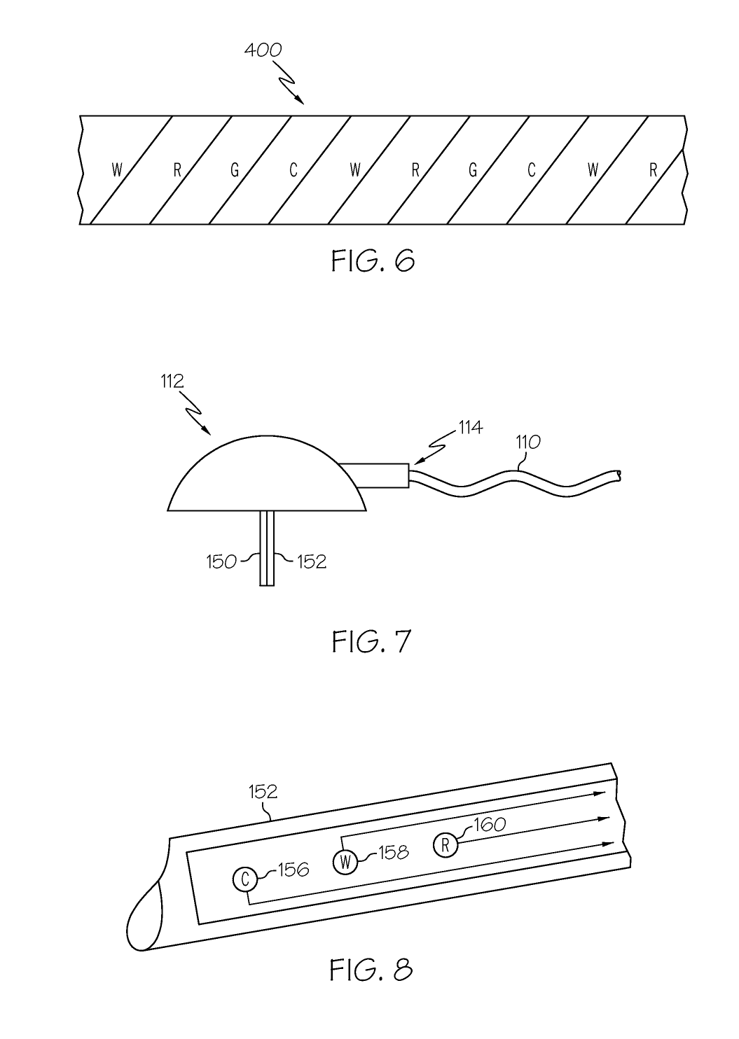

Depending upon the particular implementation of the tube 110, it may be desirable to arrange the sensor conductors along the length of the tube 110 in accordance with a predetermined winding, braiding, or twisting scheme. Twisting or braiding may be desirable to reduce inductive interference and/or to otherwise address electromagnetic phenomena associated with the sensor conductors. In this regard, the tube 110 and its tubing material have an overall length corresponding to the major longitudinal dimension. In other words, the overall length of the tube 110 is defined between the combined infusion-sensor unit 112 and the connector assembly 116 (see FIG. 1). In certain embodiments, the sensor conductors are twisted along the length of the tubing material, as depicted in FIG. 6, which is a diagram that illustrates a twisted arrangement of sensor conductors along the length of an infusion set tube 400. The labels W, R, G, and C represent the four different sensor conductors associated with the work, reference, ground, and counter electrodes, as described above. For this twisted arrangement, each sensor conductor spirals around the tube 400 (either embedded within the tubing material as described above for the tube 110, or around the exterior of the tubing material as described above for the tube 200). FIG. 6 depicts an implementation where none of the sensor conductors overlap. In alternate implementations, however, the sensor conductors may overlap one another in a braided arrangement.

FIG. 7 is a side view of an exemplary embodiment of the combined infusion-sensor unit 112. As mentioned above, the combined infusion-sensor unit 112 is coupled to the end 114 of the tube 110 and to the sensor conductors carried by the tube 110. This arrangement enables the combined infusion-sensor unit 112 to accommodate delivery of fluid from the interior fluid canal 132 of the tube 110, and to accommodate sensing of the analyte of the patient. As schematically depicted in FIG. 7, the combined infusion-sensor unit 112 includes a cannula port 150 and a sensor element 152. In certain embodiments, the sensor element 152 is integrated with the cannula port 150. The cannula port 150 is in fluid communication with the interior fluid canal 132 of the tube 110. Thus, the cannula port 150 is used to deliver fluid to the body of the patient when the cannula port 150 is properly inserted. The sensor element 152 includes the sensor electrodes that are used to detect the monitored analyte of the patient when the sensor element is properly inserted. In this regard, FIG. 8 is a schematic representation of electrodes of an analyte sensor suitable for use with the combined infusion-sensor unit 112. The sensor element 152 shown in FIG. 8 includes a counter electrode 156, a working electrode 158, and a reference electrode 160. As explained above, the counter electrode 156 is electrically coupled to the counter conductor integrated in the tube 110, the working electrode 158 is electrically coupled to the working conductor integrated in the tube 110, and the reference electrode 160 is electrically coupled to the reference conductor integrated in the tube 110.

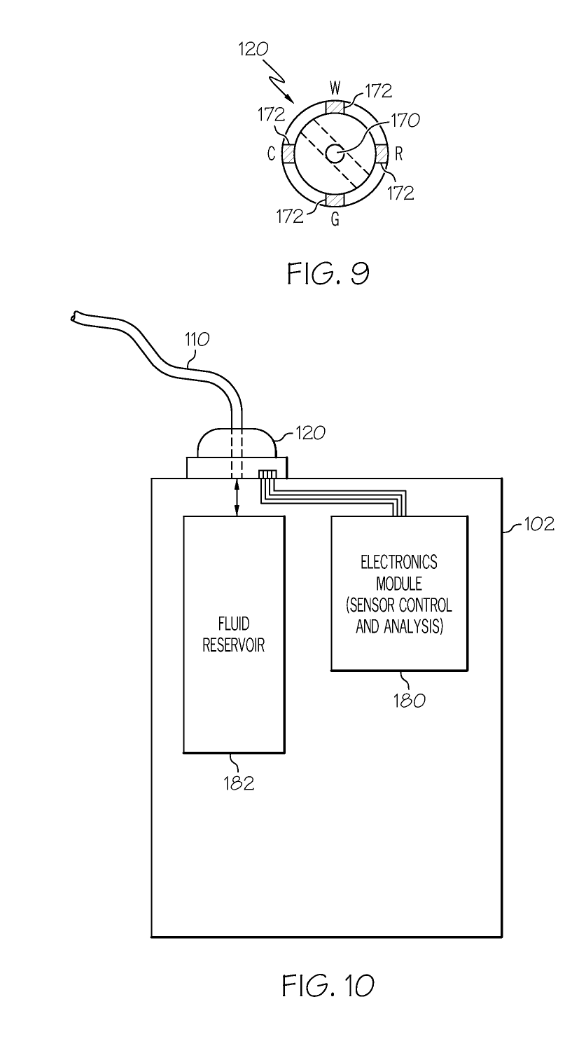

As mentioned previously with reference to FIG. 1, the other end 118 of the tube 110 may be terminated with a suitably configured connector assembly 116. The connector assembly 116 shown in FIG. 1 is realized as a removable reservoir cap 120 that cooperates with the fluid infusion device 102 and with the fluid reservoir installed in the fluid infusion device 102. In this regard, FIG. 9 is a schematic representation of electrical contacts of the reservoir cap 120. FIG. 9 may correspond to an end view or a cross-sectional view of the reservoir cap 120. The reservoir cap 120 may include a port needle 170 (and/or other features) that fluidly couples the fluid reservoir to the interior fluid canal 132 of the tube 110. The reservoir cap 120 is also configured to electrically couple the sensor conductors to an electronics module of the fluid infusion device 102. Accordingly, the sensor conductors may terminate at the reservoir cap 120. In practice, the reservoir cap 120 may include electrical contacts or terminations that correspond to the sensor conductors. FIG. 9 depicts four electrical contacts 172 incorporated into the reservoir cap 120; these electrical contacts 172 correspond to the three sensor conductors (counter, working, and reference) and to an optional ground conductor. When the reservoir cap 120 is secured to the fluid infusion device 102, the electrical contacts 172 are aligned with, and establish electrical connections with, corresponding electrical terminals or contacts of the fluid infusion device 102 (which in turn are connected to the electronics module).

The electrical contacts 172, the reservoir cap 120, and the sensor conductors integrated in the tube 110 enable the fluid infusion device 102 to apply sensing voltage to sensor conductors as needed. In practice, an electronics module of the fluid infusion device 102 may be used to generate voltage, current, and/or electrical signals for the sensor element 152, and the electronics module may also be used to detect voltage, current, resistance, capacitance, and/or electrical signals (produced by the sensor element) that indicate certain characteristics of the analyte being monitored. In this regard, the embodiment of the fluid infusion device 102 shown in FIG. 10 includes an electronics module 180 that is electrically connected to the contacts or terminals of the reservoir cap 120. The electronics module 180 is responsible for sensor control and analysis. In practice, the electronics module 180 may also be responsible for other features, operations, and functions of the fluid infusion device 102. FIG. 10 also illustrates a fluid reservoir 182 installed in the fluid infusion device 102. The fluid reservoir 182 is fluidly coupled to the tube 110, as described above.

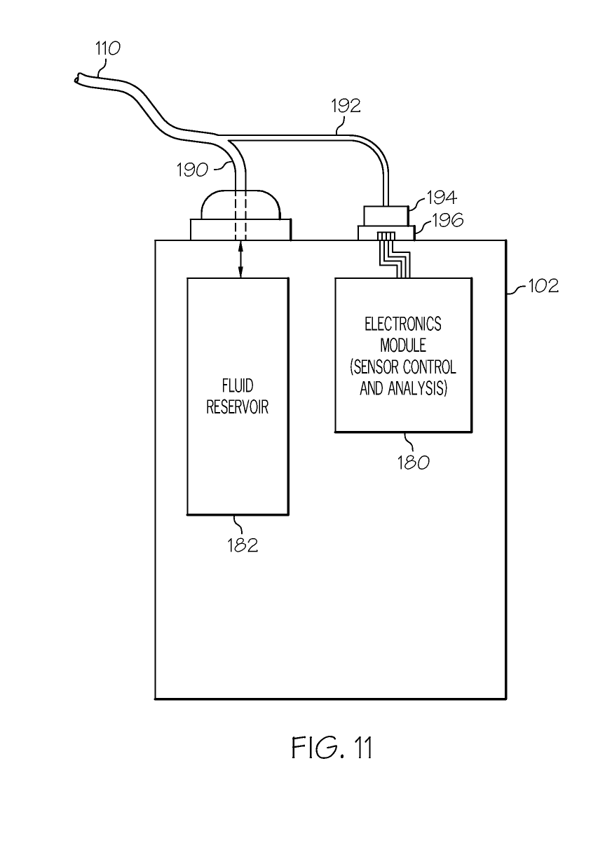

In accordance with an alternate embodiment, the tube 110 may employ a bifurcated connector assembly that has one coupling element for the interior fluid canal 132 and another coupling element for the electrical conductors integrated in the tube 110. FIG. 11 depicts an exemplary embodiment that employs two distinct coupling elements for the fluid infusion device 102. As shown in FIG. 11, the tube 110 splits into two portions: a fluid section 190 and an electrical section 192. The fluid section 190 is in fluid communication with both the interior fluid canal 132 and the fluid reservoir 182. The electrical conductors are routed from the tube to the electrical section 192, which terminates at an electrical connector 194. The electrical connector 194 mates with a corresponding electrical interface feature 196 of the fluid infusion device 102, such as a plug, a port, or a socket. Thus, the electrical connector 194 electrically couples the sensor conductors to the electrical interface feature 196 (when the two are mated together), which in turn is electrically coupled to the electronics module 180 of the fluid infusion device 102. A split connector assembly may be desirable in certain applications that have certain manufacturing or packaging requirements or challenges.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application.

What is claimed is:

1. An infusion set component for a fluid infusion device that delivers fluid to a patient, the infusion set component comprising:
tubing material having an interior fluid canal defined therein to provide a fluid pathway from the fluid infusion device to the patient; and
a plurality of sensor conductors integrated with the tubing material to facilitate sensing of an analyte of the patient by the fluid infusion device.

2. The infusion set component of claim 1, the plurality of sensor conductors comprising:
a reference conductor for a reference electrode of an analyte sensor;
a working conductor for a working electrode of the analyte sensor; and
a counter conductor for a counter electrode of the analyte sensor.

3. The infusion set component of claim 1, the plurality of sensor conductors being embedded in the tubing material.

4. The infusion set component of claim 3, wherein:
the tubing material comprises an inner tube and an outer tube concentric with and surrounding the inner tube; and
the plurality of sensor conductors are embedded in the outer tube.

5. The infusion set component of claim 3, the tubing material being composed of an electrically insulating material to electrically insulate each of the plurality of sensor conductors.

6. The infusion set component of claim 1, wherein:
the tubing material has a length corresponding to its major dimension; and
the plurality of sensor conductors are twisted along the length of the tubing material.

7. The infusion set component of claim 1, wherein:
the tubing material has a length corresponding to its major dimension; and
the plurality of sensor conductors are braided along the length of the tubing material.

8. An infusion set component for a fluid infusion device that delivers fluid to a patient, the infusion set component comprising:
a tube formed from tubing material having an interior fluid canal defined therein to provide a fluid pathway from the fluid infusion device to the patient;
a plurality of sensor conductors incorporated with the tubing material to facilitate sensing of an analyte of the patient by the fluid infusion device; and
a combined infusion-sensor unit coupled to the tube and to the plurality of sensor conductors, the combined infusion-sensor unit accommodating delivery of fluid from the interior fluid canal of the tube and accommodating sensing of the analyte.

9. The infusion set component of claim 8, the plurality of sensor conductors comprising:
a reference conductor for a reference electrode of the combined infusion-sensor unit;
a working conductor for a working electrode of the combined infusion-sensor unit; and
a counter conductor for a counter electrode of the combined infusion-sensor unit.

10. The infusion set component of claim 8, the plurality of sensor conductors being embedded in the tubing material.

11. The infusion set component of claim 8, wherein:
the tube comprises an inner tube and an outer tube concentric with and surrounding the inner tube; and
the plurality of sensor conductors are embedded in the tubing material of the outer tube.

12. The infusion set component of claim 8, wherein:
the tube has a length corresponding to its major dimension; and
the plurality of sensor conductors are twisted along the length of the tube.

13. The infusion set component of claim 8, wherein:
the tube has a length corresponding to its major dimension; and
the plurality of sensor conductors are braided along the length of the tube.

14. The infusion set component of claim 8, wherein:
the combined infusion-sensor unit is coupled to a first end of the tube; and
the infusion set component further comprises a connector assembly coupled to a second end of the tube, the connector assembly being configured to fluidly couple the interior fluid canal to a fluid reservoir of the fluid infusion device and to electrically couple the plurality of sensor conductors to an electronics module of the fluid infusion device.

15. An infusion set component for a fluid infusion device that delivers fluid to a patient, the infusion set component comprising:
a tube formed from tubing material having an interior fluid canal defined therein to provide a fluid pathway from the fluid infusion device to the patient;
a plurality of sensor conductors molded within in the tubing material to facilitate sensing of an analyte of the patient by the fluid infusion device; and
a connector assembly coupled to the tube and to the plurality of sensor conductors, the connector assembly configured to fluidly couple the interior fluid canal to a fluid reservoir of the fluid infusion device and to electrically couple the plurality of sensor conductors to an electronics module of the fluid infusion device.

16. The infusion set component of claim 15, wherein:
the connector assembly is coupled to a first end of the tube; and
the infusion set component further comprises a combined infusion-sensor unit coupled to a second end of the tube, the combined infusion-sensor unit accommodating delivery of fluid from the interior fluid canal of the tube and accommodating sensing of the analyte.

17. The infusion set component of claim 16, the plurality of sensor conductors comprising:
a reference conductor for a reference electrode of the combined infusion-sensor unit;
a working conductor for a working electrode of the combined infusion-sensor unit; and
a counter conductor for a counter electrode of the combined infusion-sensor unit.

18. The infusion set component of claim 16, wherein:
the tube has a length corresponding to its major dimension; and
the plurality of sensor conductors are twisted along the length of the tube.

19. The infusion set component of claim 16, wherein:
the tube has a length corresponding to its major dimension; and
the plurality of sensor conductors are braided along the length of the tube.

20. The infusion set component of claim 15, wherein the connector assembly comprises a reservoir cap for the fluid reservoir, the plurality of sensor conductors terminating at the reservoir cap.

21. The infusion set component of claim 15, wherein the connector assembly comprises:
a reservoir cap for the fluid reservoir, the reservoir cap fluidly coupling the interior fluid canal to the fluid reservoir; and
an electrical connector that mates with an electrical interface feature of the fluid infusion device, the electrical connector electrically coupling the plurality of sensor conductors to the electrical interface feature.

22. The infusion set component of claim 15, wherein the connector assembly enables the fluid infusion device to apply sensing voltage to the plurality of sensor conductors.

23. The infusion set component of claim 15, wherein:
the tube comprises an inner tube and an outer tube concentric with and surrounding the inner tube; and
the plurality of sensor conductors are molded within the tubing material of the outer tube.
